(19) United States
(12) Patent Application Publication
(10) Pub. No.: US 2025/0127150 A1
(43) Pub. Date: Apr. 24, 2025

(54) UNMANNED INSECT BREEDING SYSTEM AND METHOD OF OPERATING THE SAME

(71) Applicant: VANDALSOFT INC., Seongnam-Si (KR)

(72) Inventor: Bong Hak Lee, Daejeon (KR)

(73) Assignee: VANDALSOFT INC., Seongnam-Si (KR)

(21) Appl. No.: 18/288,608

(22) PCT Filed: Aug. 17, 2023
(86) PCT No.: PCT/KR2023/012156

§ 371 Date: Oct. 27, 2023

(30) Foreign Application Priority Data
Jul. 26, 2023 (KR) 10-2023-0097223

(51) Int. Cl. A01K 67/362; A01K 67/368; B25J 9/02; B25J 15/02; B65G 1/04

(52) U.S. Cl. CPC A01K 67/362; A01K 67/368; B25J 9/026; B25J 15/0253; B65G 1/0478

(57) ABSTRACT

The present invention relates to an unmanned insect breeding system. The unmanned insect breeding system includes a feed tank configured to store a feed for insects, an insect farm in which box stacks in which a plurality of boxes containing insects are vertically stacked are arranged in m rows and n columns, an automatic feeding device configured to supply the feed in the feed tank to the boxes, an insect sorting device disposed at one side of the insect farm and including a sorter configured to sort the insects, a guide rail installed in front of the insect farm and extending in a width direction of the insect farm, a box transfer robot which moves forward and rearward on the guide rail and loads and unloads the box stack, and a control unit configured to control driving of the box transfer robot.

### TECHNICAL FIELD

The present invention relates to an unmanned insect breeding system which is a system having a multi-layered structure that breeds insects in an automated manner and is capable of efficiently using a space and achieving high productivity, and a method of operating the same.

### BACKGROUND ART

Currently, when agrifood by-products such as food waste discharged from homes, restaurants, and the like, organic waste generated at industrial sites, and organic waste such as livestock excretions and human excretions generated from livestock farms are discharged without treatment, it may cause serious environmental problems in soil, rivers, and the like.

As conventional methods, a method of recycling organic waste into livestock feed or compost through a specific treatment process and a method of landfilling organic waste in a certain area or dumping the organic waste in the ocean have been mainly used. In this case, there is a problem of low economic feasibility in terms of installation and maintenance costs of a treatment device. In addition, when the discharge is used as feed without any change, there are significant side effects such as the outbreak of mysterious diseases, and even in the case of composting, there are problems such as reheating and an imbalance in fertilizer effect.

In addition, in order to solve the above problems, an organic waste treatment system using living organisms such as earthworms and housefly larvae has been developed. In this case, it is known that the excreted fecal soil may have an excellent fertilizer effect to be usefully used for cultivation in agricultural lands, horticulture farms, orchards, and the like and may serve to increase the growth of plants and the yield of products.

Among these, it takes 37 to 41 days for soldier flies to grow from egg to mature adult, and it takes about 14 days for larvae, which act as decomposers, to grow from egg to mature adult. After a period (about 14 days) during which soldier fly larvae can decompose organic waste, the soldier fly larvae have a habit of crawling out to find a dry place to pupate. When 5,000 soldier fly larvae are introduced into 10 kg of food waste, 80% or more of the food waste is decomposed in just 5 days, and the decomposed food waste is reduced in volume by about 42% and weight by about 70% as compared with before the introduction of the larvae. Soldier fly larvae have a longer period of time to decompose organic waste resources than earthworms or houseflies, and thus it is much more effective to breed and utilize soldier fly larvae.

However, organic waste treatment devices using earthworms or flies have a problem such as being applicable only to a small amount of organic waste generated in homes, a problem of bad odor generated when organic waste decays, and a problem of difficulty in separating feces and larvae.

Therefore, fly larvae can not only process organic waste but can also be used for animal feed and oil extraction, and there is a need to develop an automatic collection device for old larvae that can efficiently breed and multiply the fly larvae. Therefore, there is a need to develop an automated treatment device that can save labor by developing an automatic soldier larvae breeding device capable of continuously breeding soldier larvae and automatically collecting old larvae.

### RELATED ART DOCUMENTS

### Patent Documents

(Patent Document 1) Korean Patent Registration No. 10-1256389 (published on 25 Apr. 2013)

### DISCLOSURE

### Technical Problem

The present invention is directed to providing an unmanned insect breeding system which automatically breeds insects in the form of a box stack in which a plurality of boxes are vertically stacked, is operable in an unmanned manner through a transfer robot, and simultaneously allows a breeding device and a sorting device to be integrated.

### Technical Solution

According to an embodiment of the present invention, an unmanned insect breeding system includes a feed tank configured to store a feed for insects, an insect farm in which box stacks in which a plurality of boxes containing insects are vertically stacked are arranged in m rows and n columns, an automatic feeding device configured to supply the feed in the feed tank to the boxes, an insect sorting device disposed at one side of the insect farm and including a sorter configured to sort the insects, a guide rail installed in front of the insect farm and extending in a width direction of the insect farm, a box transfer robot which moves forward and rearward on the guide rail and loads and unloads the box stack, and a control unit configured to control driving of the box transfer robot, wherein the insect farm includes a rearward movement row in which the box stacks are pushed rearward one space at a time every set number of days and moved and a forward movement row in which the box stacks are pushed forward one space at a time every set number of days and returned, and the control unit moves the box transfer robot forward or rearward on the guide rail and transfers the box stack between the insect sorting device, the rearward movement row, and the forward movement row.

The insect sorting device may further include a stack temporary storage unit in which the box stack transferred from the insect farm by the box transfer robot is temporarily placed, and an axis transfer robot configured to pick up the boxes from both the stack temporary storage unit and the sorter and transfer the boxes along an x-axis and a z-axis, and the control unit may control driving of the axis transfer robot.

The unmanned insect breeding system may further include an x-axis transfer rail configured to support and move the axis transfer robot forward or rearward along an x-axis, and the axis transfer robot may include a gripper configured to pick up an uppermost box of the box stack in the stack temporary storage unit and a box rotation portion configured to rotate the picked up box and shake the insects in the box into the sorter.

A coupling groove into which the gripper is coupled may be formed in each box in the box stack, the gripper may include two gripper bodies, a fitting protrusion fitted into the coupling groove of the box, and a gripper motor configured to adjust a separation distance between the two gripper bodies, and the axis transfer robot may lower the gripper to lower the fitting protrusion to a level of the coupling groove and then may narrow an interval between the two gripper bodies through the gripper motor to pick up the box.

The sorting device may include a sorting plate formed in a net shape to sort and separate the insects from foreign materials, a vibration motor configured to vibrate the sorting plate, an insect storage container disposed below a side of the sorting plate to contain the selected insects, and a tilt adjustment unit configured to adjust a front end height of the sorting plate to adjust a tilt of the sorting plate so that the insects sorted on the sorting plate are put into the insect storage container.

The stack temporary storage unit may have a first space, into which the box stack in which breeding is completed in the insect farm is put, and a second space in which the box stack which is refilled with the insects after a sorting operation and is to be put again into the insect farm is placed, and the axis transfer robot may transfer the box stack in the first space to the sorter to allow the sorting operation to be performed and may transfer the box stack refilled with the insects to the second space after the sorting operation.

The box transfer robot may include a housing which engages with the guide rail and moves forward or rearward on the guide rail, and a box loading unit which supports and loads the box stack by moving relative to the housing.

The box stack may be loaded on a tray equipped with a plurality of wheels, and when the box transfer robot pushes the tray into the rearward movement row or the forward movement row, the box stacks may be pushed one space at a time and moved.

The guide rail and box transfer robot may include a first rail and a first box transfer robot which are disposed in front of the insect farm, and a second rail and a second box transfer robot which are disposed behind the insect farm, and the control unit may control the first box transfer robot to pick up the box stack of the stack temporary storage unit to transfer and push the box stack into the rearward movement row and pick up the box stack of the forward movement row to transfer and push the box stack into the stack temporary storage unit.

The control unit may control the second box transfer robot to pick up an endmost box stack in the rearward movement row to transfer and push the box stack into the forward movement row.

When the axis transfer robot picks up the uppermost box of the box stack of the stack temporary storage unit and transfers the box to the sorter, the control unit may put the insects in the box into the sorter through the box rotation unit, and after a sorting operation, may put down the box stack on the stack temporary storage unit.

The insect sorting device may further include an air blower configured to blow air to clean the box emptied after the sorting operation, the air blower may include a housing configured to accommodate the empty box and an air pump configured to blow air into the housing, and the control unit may cause the axis transfer robot to transfer the empty box into the air blower, perform cleaning, and then transfer the empty box to the stack temporary storage unit.

According to another embodiment of the present invention, a method of operating an unmanned insect breeding system is an unmanned insect breeding method using an unmanned insect breeding system which includes an insect farm in which box stacks in which a plurality of boxes containing insects are vertically stacked are arranged in m rows and n columns, an insect sorting device including an insect temporary storage unit and an axis transfer robot, and a box transfer robot, wherein the insect farm includes a rearward movement row in which the box stacks are pushed rearward one space at a time every set number of days and moved and a forward movement row in which the box stacks are pushed forward one space at a time every set number of days and returned, and the box transfer robot includes a first box transfer robot disposed in front of the insect farm and a second box transfer robot disposed behind the insect farm, the method including operation a) of picking up, by the first box transfer robot, the box stack of the stack temporary storage unit to transfer and push the box stack into the rearward movement row, operation b) of picking up, by the second box transfer robot, an endmost box stack in the rearward movement row to transfer and push the box stack into the forward movement row, and operation c) of picking up, by the axis transfer robot in the insect sorting device, an uppermost box of the box stack of the stack temporary storage unit to transfer the box to the sorter and put the insects in the box into the sorter, and putting down the box after a sorting operation on the stack temporary storage unit.

The insect sorting device may further include an air blower configured to blow air to clean the empty box, and operation c) may include transferring, by the axis transfer robot, the empty box into the air blower to perform cleaning, and then transferring the empty box to the stack temporary storage unit.

The stack temporary storage unit may have a first space, into which the box stack in which breeding is completed in the insect farm is put, and a second space in which the box stack which is refilled with the insects after the sorting operation and is to be put again into the insect farm is placed, and operation c) may include operation (c-1) of transferring, by the axis transfer robot, the box of the box stack in the first space to the sorter to perform the sorting operation, and operation (c-2) of transferring, by the axis transfer robot, the box stack, which is refilled with the insects after the sorting operation, to the second space.,

### Advantageous Effects

The effects of the present invention are as follows.

1. Automated insect breeding: Previously, manually breeding insects has been a cumbersome and time-consuming task. However, through the present invention, insects can be automatically bred in the form of a stack, thereby saving manpower and time.

2. Unmanned operation: A transfer robot can be used to manage and move box stacks containing insects, thereby performing an unmanned operation. This allows breeding and management of insects without operator participation.

3. Efficient insect sorting: A breeding device can be integrated with a sorting device, thereby sorting bred insects. Thus, insects can be sorted from foreign materials, and the sorted insects can be used as feed or the like.

4. Increased productivity: Due to an unmanned breeding system, manpower and time can be saved, and efficient management can be performed, thereby increasing productivity. For example, waste can be efficiently treated by breeding large quantities of organic waste treatment larvae such as soldier flies.

### DESCRIPTION OF DRAWINGS

FIG. 1 is a perspective view of the overall configuration of an unmanned insect breeding system according to the present invention.

FIG. 2 is a plan view of the unmanned insect breeding system of FIG. 1.

FIG. 3 is a side view of the unmanned insect breeding system of FIG. 1.

FIG. 4 is a front view of the unmanned insect breeding system of FIG. 1.

FIG. 5 is a perspective view of an insect sorting device in the unmanned insect breeding system of FIG. 1.

FIG. 6 is a view illustrating a sorter of the insect sorting device of FIG. 5.

FIG. 7 is a perspective view illustrating a process in which a box stack is transferred from an insect farm and put into a stack temporary storage unit of the insect sorting device of FIG. 5.

FIG. 8 is a perspective view illustrating a process of placing a box stack refilled with insects after a sorting operation in FIG. 7.

FIG. 9 is a view illustrating an axis movement robot of the insect sorting device of FIG. 5.

### MODES OF THE INVENTION

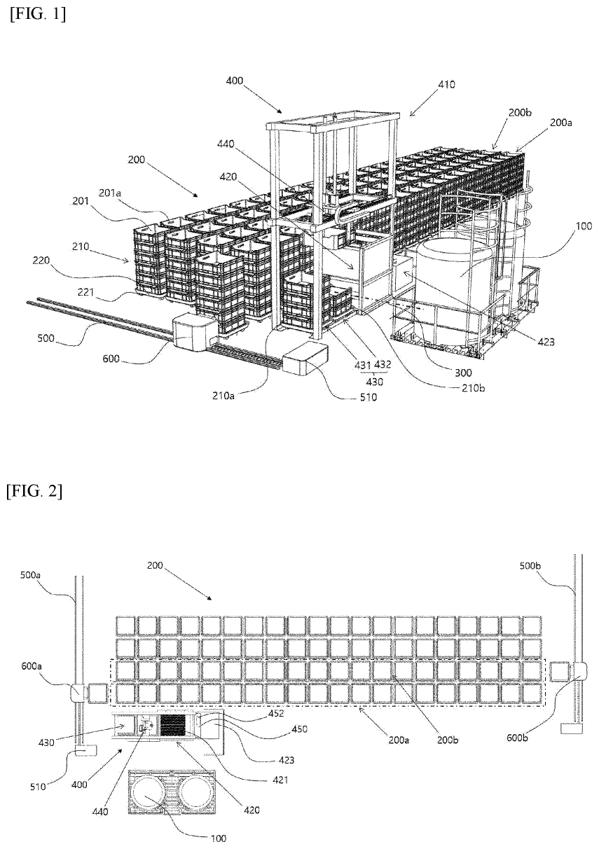

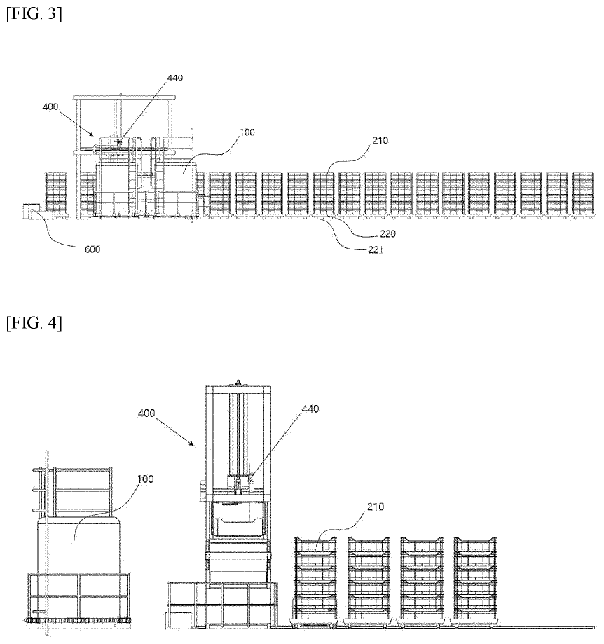

Hereinafter, specific contents for implementing the present invention will be described with reference to the accompanying drawings. In addition, in description of the present invention, when it is determined that detailed description of related well-known functions which are clear to those skilled in the art may unnecessarily obscure the gist of the present invention, such detailed description will be omitted. FIG. 1 is a perspective view of the overall configuration of an unmanned insect breeding system according to the present invention. FIG. 2 is a plan view of the unmanned insect breeding system of FIG. 1. FIG. 3 is a side view of the unmanned insect breeding system of FIG. 1. FIG. 4 is a front view of the unmanned insect breeding system of FIG. 1.

Referring to FIGS. 1 to 4, the unmanned insect breeding system according to the present invention includes a feed tank 100, an insect farm 200, an automatic feeding device 300, an insect sorting device 400, a guide rail 500, a box transfer robot 600, and a control unit. Insects to be bred may be any insects such as soldier flies and crickets.

An insect feed is stored in the feed tank 100. For example, when the insects to be bred are crickets, the insect feed may be a cricket feed and when the insects to be bred are soldier flies, the insect feed may be food waste or the like.

In the insect farm 200, a plurality of box stacks 210 are arranged in m rows and n columns. The box stack 210 has a configuration in which the plurality of boxes 201 containing insects are stacked vertically. The box stack 210 is loaded on a tray 220 equipped with a plurality of wheels 221. In the insect farm 200, the box stacks 210 are transferred in a c-shape to be put one by one into a rearward movement row 200a and exit to a forward movement row 200b.

In the rearward movement row 200a, the box stacks 210 are pushed rearward one space at a time every set number of days and moved. In the forward movement row 200b, the box stacks 210 are pushed forward one space at a time every set number of days and returned. The set number of days is set to, for example, 1 day. That is, one or two box stacks 210 are newly put into the rearward movement row 200a for one day. For half of the total breeding days, breeding is performed in the rearward movement row 200a, and for the other half thereof, breeding is performed in the forward movement row 200b.

The automatic feeding device 300 supplies the feed in the feed tank 100 to the boxes 201. One side of the automatic feeding device 300 is connected to the feed tank 100, and the other side thereof extends to the box stacks 210 which are placed in a stack temporary storage unit 430 after sorting is completed in the insect sorting device 400. The automatic feeding device 300 includes a pipe and a spray nozzle, and the feed in the feed tank 100 is supplied to the box stacks 210, in which the sorting is completed, through the pipe and the spray nozzle.

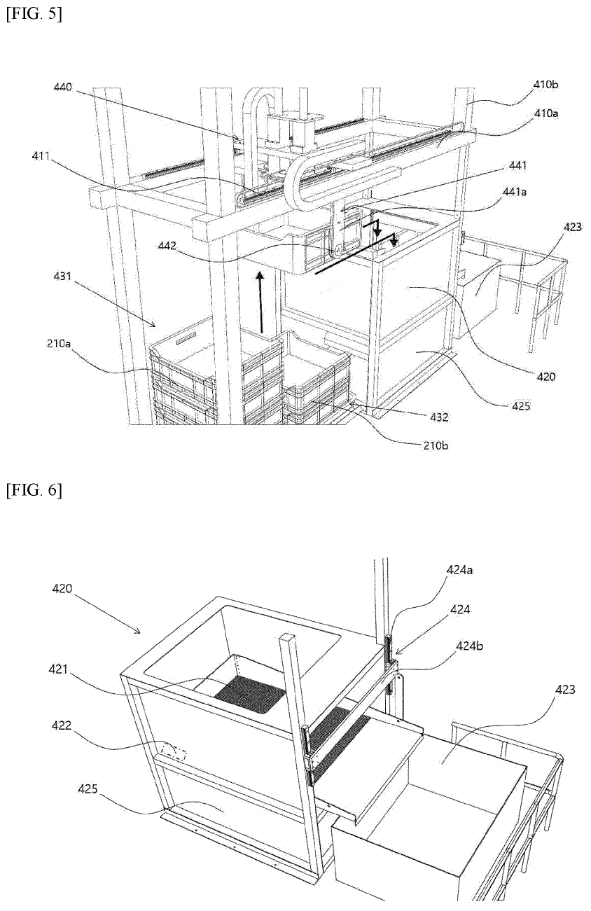

FIG. 5 is a perspective view of the insect sorting device in the unmanned insect breeding system of FIG. 1. FIG. 6 is a view illustrating a sorter of the insect sorting device of FIG. 5.

Referring FIGS. 5 and 6, the insect sorting device 400 includes a frame 410, a sorter 420, the stack temporary storage unit 430, an axis transfer robot 440, and an air blower 450. The insect sorting device 400 is disposed at one side of the insect farm 200. The frame 410 includes a horizontal frame 410a and a vertical frame 410b. Among these, an x-axis transfer rail 411 that is disposed above the sorter 420 and extends horizontally is installed on the horizontal frame 410a. The axis transfer robot 440 is supported on the x-axis transfer rail 411 to move forward or rearward along an x-axis.

The sorter 420 includes a sorting plate 421, a vibration motor 422, an insect storage container 423, a tilt adjustment unit 424, and a collection container 425. After breeding, the box 201 is in a state in which insects and foreign materials are mixed therein, and the sorter 420 serves to sort insects having a certain size or more from the foreign materials. The sorting plate 421 is formed in a net shape to sort and separate the insects from the foreign materials. The vibration motor 422 vibrates the sorting plate 421 so that the insects remain on the sorting plate 421 and the foreign materials pass through the sorting plate 421 and fall into the collection container 425.

The insect storage container 423 has a space for containing the sorted insects and is disposed below a side of the sorting plate 421. The tilt adjustment unit 424 adjusts the tilt of the sorting plate 421 by adjusting the front end height of the sorting plate 421. Accordingly, the insects that have been sorted on the sorting plate 421 are put into the insect storage container 423. The tilt adjustment unit 424 includes two tilt adjustment guide rails 424a provided at both front sides of the sorter 420, and a tilt adjustment guide portion 424b moving along the tilt adjustment guide rail 424a and coupled to a front end of the sorting plate 421.

The box stack 210 transferred from the insect farm 200 by the box transfer robot 600 is temporarily placed in the stack temporary storage unit 430. The stack temporary storage unit 430 includes a first space 431, into which the box stack 210a, in which breeding is completed in the insect farm 200, is put, and a second space 432 in which the box stack 210b, which is refilled with insects after a sorting operation and is to be put again into the insect farm, is placed.

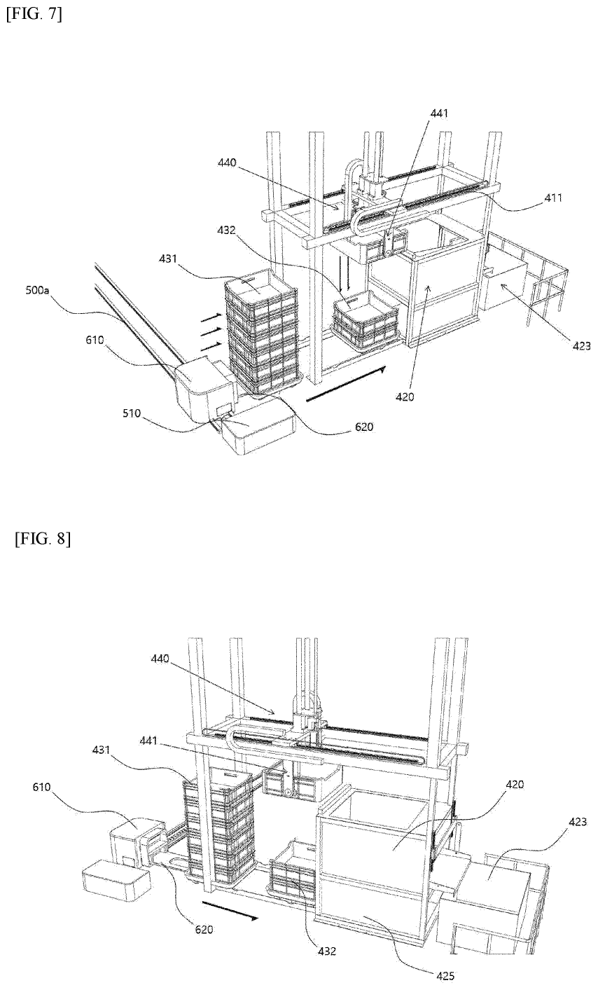

FIG. 7 is a perspective view illustrating a process in which the box stack is transferred from the insect farm and put into the stack temporary storage unit of the insect sorting device of FIG. 5. FIG. 8 is a perspective view illustrating a process of placing the box stack refilled with insects after a sorting operation in FIG. 7.

Referring to FIGS. 7 and 8, the box transfer robot 600 puts the box stack 210a, in which breeding is completed in the insect farm 200, into the first space 431 of the stack temporary storage unit 430. The axis transfer robot 440 transfers the box stack 210a in the first space 431 to the sorter 420 to allow a sorting operation to be performed. The axis transfer robot 440 transfers the box stack 210b refilled with insects to the second space 432 after a sorting operation.

The stack temporary storage unit 430 is provided with a rail device 433 that moves the box stack 210b placed in the second space 432 to the first space 431. The rail device 433 includes an electric motor for the movement. The box stack 210b that has moved to the first space 431 is picked up by the box transfer robot 600 and transferred to the insect farm 200.

The axis transfer robot 440 may pick up boxes from both the stack temporary storage unit 430 and the sorter 420 and transfer the boxes along an x-axis and a z-axis. The axis transfer robot 440 moves forward or rearward along the x-axis transfer rail 411. The axis transfer robot 440 includes a gripper 441, a box rotation portion 442, and a z-axis lift 443. The gripper 441 picks up the uppermost box 201 of the box stack 210a in the first space 431 of the stack temporary storage unit 430. The axis transfer robot 440 moves toward the sorter 420 along the x-axis transfer rail 411. The gripper 441 rotates the picked up box 201 to put a mixture of insects and foreign materials in the box 201 into the sorter 420.

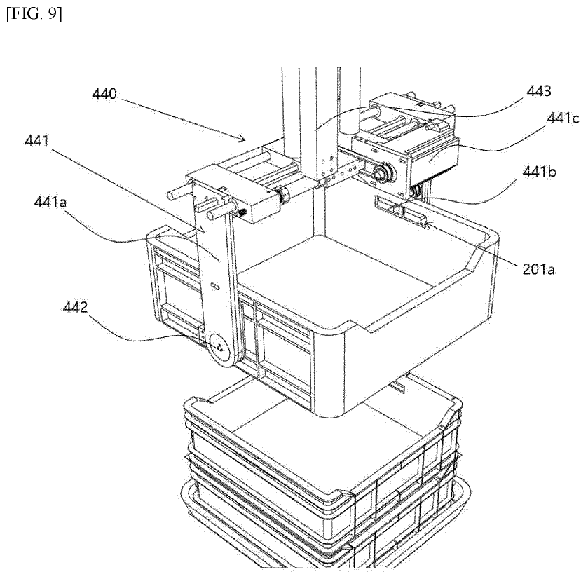

FIG. 9 is a view illustrating an axis movement robot of the insect sorting device of FIG. 5.

Referring to FIG. 9, a coupling groove 201a to which the gripper 441 may be coupled is formed in each box 201 in the box stack 210. The gripper 441 includes two gripper bodies 441a, a fitting protrusion 441b that may be fitted into the coupling groove 201a of the box 201, and a gripper motor 441c that adjusts a separation distance between the two gripper bodies 441a. The gripper bodies 441a are coupled to both sides of a body of the axis transfer robot 440 and each extend downward. The fitting protrusion 441b is formed on an inner surface of the gripper body 441a to have a size that fits into the coupling groove 201a.

The axis transfer robot 440 lowers the gripper 441 to allow the fitting protrusion 441b to be placed at a level of the coupling groove 201a of the uppermost box 201 of the box stack 210. Next, the gripper motor 441c operates to narrow an interval between the two gripper bodies 441a to allow the fitting protrusion 441b to be fitted into the coupling groove 201a, and thus the box 201 is picked up.

The box rotation portion 442 is provided to interlock with the fitting protrusion 441b in the gripper body 441a. When the box rotation portion 442 is rotated by a rotation motor, the box picked up by the gripper 441 is turned over. Thus, all insects and foreign materials can be emptied out of the box 201. The z-axis lift 443 controls the lifting or lowering of the gripper 441 in the axis transfer robot 440.

When the axis transfer robot 440 picks up the uppermost box of the box stack 210 of the stack temporary storage unit 430 and transfers the uppermost box to the sorter 420, the control unit shakes the insects in the box 201 into the sorter 420 through the box rotation portion 442. After a sorting operation, the axis transfer robot 440 puts down the box stack 210 on the stack temporary storage unit 430.

The air blower 450 blows air to clean the box emptied after the sorting operation. The air blower 450 includes a housing 451 that may accommodate the empty box, and an air pump 452 that blows air into the housing 451. The control unit performs control such that the axis transfer robot 440 transfers the empty box into the air blower 450, performs cleaning, and then transfers the empty box to the stack temporary storage unit 430. As a result, after insects are cleaned, the empty box may also be cleaned and returned to its initial state, and thus insects and a feed may be put again into the empty box.

The guide rail 500 is installed in front of the insect farm 200 and extends in a width direction of the insect farm 200. The guide rail 500 serves to guide the movement of the box transfer robot 600. The guide rail 500 includes a first rail 500a disposed in front of the insect farm 200, and a second rail 500b disposed behind the insect farm 200. A motor 510 that controls the movement of the box transfer robot 600 on the rail is installed on the guide rail 500.

The box transfer robot 600 may move forward or rearward on the guide rail 500 to load and unload the box stack 210. The box transfer robot 600 includes a housing 610 and a box loading unit 620. The housing 610 engages with the guide rail 500 and moves forward or rearward on the guide rail 500. The box loading unit 620 may support and load the box stack 210 by moving relative to the housing 610. The box loading unit 620 extends from the housing 610, supports the tray 220, on which the box stack 210 is loaded, and then pulls the tray 220 to pick up the box stack 210.

When the box transfer robot 600 pushes the tray 220 into the rearward movement row 200a or the forward movement row 200b, the stacks 210 are pushed one space at a time and moved. The box transfer robot 600 includes a first box transfer robot 600a disposed in front of the insect farm 200 and moving along the first rail 500a, and a second box transfer robot 600b disposed behind the insect farm 200 and moving along the second rail 500b.

The control unit controls the driving of the axis transfer robot 440 and the box transfer robot 600. The control unit may move the box transfer robot 600 forward or rearward on the guide rail 500 and may transfer the box stack 210 between the insect sorting device 400, the rearward movement row 200a, and the forward movement row 200b. The control unit causes the first box transfer robot 600a to pick up the box stack 210 of the stack temporary storage unit 430 and push the box stack 210 into the rearward movement row 200a and causes the first box transfer robot 600a to pick up the box stack 210 in the forward movement row 200b and transfer and push the box stack 210 into the stack temporary storage unit 430.

The control unit causes the second box transfer robot 600b to pick up the box stack 210 of the rearward movement row 200a and transfer and push the box stack 210 into the forward movement row 200b. Thus, the box stacks 210 in which half of the entire breeding schedule has been performed in the rearward movement row 200a can be moved to the forward movement row 200b to perform breeding of the remaining half.

A method of operating an unmanned insect breeding system using the unmanned insect breeding system will be described below.

First, the first box transfer robot 600a picks up the box stack 210 of the stack temporary storage unit 430 and transfers and pushes the box stack 210 to the rearward movement row 200a of the insect farm 200. Accordingly, the box stacks 210 in the rearward movement row 200a are pushed rearward one by one. Then, the second box transfer robot 600b picks up the endmost box stack 210 in the rearward movement row 200a and transfers and pushes the box stack 210 into the forward movement row 200b.

In the insect sorting device 400, the axis transfer robot 440 picks up the uppermost box 201 of the box stack 210 in the first space 431 of the stack temporary storage unit 430, transfers the uppermost box 201 to the sorter 420, and puts insects in the box into the sorter 420. The axis transfer robot 440 refills the box after a sorting operation with insects, transfers the box to the second space 432 of the stack temporary storage unit 430, and puts down the box.

In this case, the axis transfer robot 440 transfers the empty box after the sorting operation into the air blower 450, performs cleaning, and then transfers the empty box to the stack temporary storage unit 430. The cleaned box is refilled with insects, a feed is supplied from the automatic feeding device 300, and the box is stacked in the second space 432 in the form of the box stack 210. The box stack 210 is picked up by the box transfer robot 600 and put into the rearward movement row 200a of the insect farm 200.

The scope of the present invention in the art is not limited to the content and description of the above-described embodiments. In addition, it is mentioned once again that the scope of the present invention is not limited by obvious changes or substitutions in the art to which the present invention belongs.

### DESCRIPTION OF REFERENCE NUMERALS

100: feed tank
200: insect farm
200a: rearward movement row
200b: forward movement row
201: box
201a: coupling groove
210: box stack
210a: box stack in which breeding is completed
210b: box stack refilled with insects to be reloaded
220: tray
221: wheel
300: automatic feeding device
400: insect sorting device
410: frame
410a: horizontal frame
410b: vertical frame
411: x-axis transfer rail
420: sorter
421: sorting plate
422: vibration motor
423: insect storage container
424: tilt adjustment unit
424a: tilt adjustment guide rail
424b: tilt adjustment guide portion
425: collection container
430: stack temporary storage unit
431: first space
432: second space
433: rail device
440: axis transfer robot
441: gripper
441a: gripper body
441b: fitting protrusion
441c: gripper motor
442: box rotation unit
443: z-axis lift
450: air blower
451: housing
452: air pump
430: stack temporary storage unit
500: guide rail
500a: first rail
500b: second rail
510: motor
600: box transfer robot
600a: first box transfer robot
600b: second box transfer robot
610: robot housing
620: box loading unit

1. An unmanned insect breeding system comprising:
a feed tank configured to store a feed for insects;
an insect farm in which box stacks in which a plurality of boxes containing insects are vertically stacked are arranged in m rows and n columns;
an automatic feeding device configured to supply the feed in the feed tank to the boxes;
an insect sorting device disposed at one side of the insect farm and including a sorter configured to sort the insects;
a guide rail installed in front of the insect farm and extending in a width direction of the insect farm;
a box transfer robot which moves forward and rearward on the guide rail and loads and unloads the box stack; and
a control unit configured to control driving of the box transfer robot,
wherein the insect farm includes a rearward movement row in which the box stacks are pushed rearward one space at a time every set number of days and moved and a forward movement row in which the box stacks are pushed forward one space at a time every set number of days and returned, and
the control unit moves the box transfer robot forward or rearward on the guide rail and transfers the box stack between the insect sorting device, the rearward movement row, and the forward movement row.

2. The unmanned insect breeding system of claim 1, wherein the insect sorting device further includes a stack temporary storage unit in which the box stack transferred from the insect farm by the box transfer robot is temporarily placed, and an axis transfer robot configured to pick up the boxes from both the stack temporary storage unit and the sorter and transfer the boxes along an x-axis and a z-axis, and
the control unit controls driving of the axis transfer robot.

3. The unmanned insect breeding system of claim 2, further comprising an x-axis transfer rail configured to support and move the axis transfer robot forward or rearward along the x-axis,
wherein the axis transfer robot includes a gripper configured to pick up an uppermost box of the box stack in the stack temporary storage unit and a box rotation portion configured to rotate the picked up box and shake the insects in the box into the sorter.

4. The unmanned insect breeding system of claim 3, wherein a coupling groove into which the gripper is coupled is formed in each box in the box stack,
the gripper includes two gripper bodies, a fitting protrusion fitted into the coupling groove of the box, and a gripper motor configured to adjust a separation distance between the two gripper bodies, and
the axis transfer robot lowers the gripper to lower the fitting protrusion to a level of the coupling groove and then narrows an interval between the two gripper bodies through the gripper motor to pick up the box.

5. The unmanned insect breeding system of claim 1, wherein the sorting device includes a sorting plate formed in a net shape to sort and separate the insects from foreign materials, a vibration motor configured to vibrate the sorting plate, an insect storage container disposed below a side of the sorting plate to contain the selected insects, and a tilt adjustment unit configured to adjust a front end height of the sorting plate to adjust a tilt of the sorting plate so that the insects sorted on the sorting plate are put into the insect storage container.

6. The unmanned insect breeding system of claim 2, wherein the stack temporary storage unit has a first space, into which the box stack in which breeding is completed in the insect farm is put, and a second space in which the box stack which is refilled with the insects after a sorting operation and is to be put again into the insect farm is placed, and
the axis transfer robot transfers the box stack in the first space to the sorter to allow the sorting operation to be performed and transfers the box stack refilled with the insects to the second space after the sorting operation.

7. The unmanned insect breeding system of claim 1, wherein the box transfer robot includes a housing which engages with the guide rail and moves forward or rearward on the guide rail, and a box loading unit which supports and loads the box stack by moving relative to the housing.

8. The unmanned insect breeding system of claim 2, wherein the box stack is loaded on a tray equipped with a plurality of wheels, and
when the box transfer robot pushes the tray into the rearward movement row or the forward movement row, the box stacks are pushed one space at a time and moved.

9. The unmanned insect breeding system of claim 2, wherein the guide rail and box transfer robot include a first rail and a first box transfer robot which are disposed in front of the insect farm, and a second rail and a second box transfer robot which are disposed behind the insect farm, and
the control unit controls the first box transfer robot to pick up the box stack of the stack temporary storage unit to transfer and push the box stack into the rearward movement row and pick up the box stack of the forward movement row to transfer and push the box stack into the stack temporary storage unit.

10. The unmanned insect breeding system of claim 9, wherein the control unit controls the second box transfer robot to pick up an endmost box stack in the rearward movement row to transfer and push the box stack into the forward movement row.

11. The unmanned insect breeding system of claim 3, wherein, when the axis transfer robot picks up the uppermost box of the box stack of the stack temporary storage unit and transfers the box to the sorter, the control unit puts the insects in the box into the sorter through the box rotation unit, and after a sorting operation, puts down the box stack on the stack temporary storage unit.

12. The unmanned insect breeding system of claim 11, wherein the insect sorting device further includes an air blower configured to blow air to clean the box emptied after the sorting operation,
the air blower includes a housing configured to accommodate the empty box and an air pump configured to blow air into the housing, and
the control unit causes the axis transfer robot to transfer the empty box into the air blower, perform cleaning, and then transfer the empty box to the stack temporary storage unit.

13. A method of operating an unmanned insect breeding system which is an unmanned insect breeding method using an unmanned insect breeding system which includes an insect farm in which box stacks in which a plurality of boxes containing insects are vertically stacked are arranged in m rows and n columns, an insect sorting device including an insect temporary storage unit and an axis transfer robot, and a box transfer robot, wherein the insect farm includes a rearward movement row in which the box stacks are pushed rearward one space at a time every set number of days and moved and a forward movement row in which the box stacks are pushed forward one space at a time every set number of days and returned, and the box transfer robot includes a first box transfer robot disposed in front of the insect farm and a second box transfer robot disposed behind the insect farm, the method comprising:
operation a) of picking up, by the first box transfer robot, the box stack of the stack temporary storage unit to transfer and push the box stack into the rearward movement row;
operation b) of picking up, by the second box transfer robot, an endmost box stack in the rearward movement row to transfer and push the box stack into the forward movement row; and
operation c) of picking up, by the axis transfer robot in the insect sorting device, an uppermost box of the box stack of the stack temporary storage unit to transfer the box to the sorter and put the insects in the box into the sorter, and putting down the box after a sorting operation on the stack temporary storage unit.

14. The method of claim 13, wherein the insect sorting device further includes an air blower configured to blow air to clean the empty box, and
operation c) includes transferring, by the axis transfer robot, the empty box into the air blower to perform cleaning, and then transferring the empty box to the stack temporary storage unit.

15. The method of claim 13, wherein the stack temporary storage unit has a first space, into which the box stack in which breeding is completed in the insect farm is put, and a second space in which the box stack which is refilled with the insects after the sorting operation and is to be put again into the insect farm is placed, and
operation c) includes operation (c-1) of transferring, by the axis transfer robot, the box of the box stack in the first space to the sorter to perform the sorting operation, and operation (c-2) of transferring, by the axis transfer robot, the box stack, which is refilled with the insects after the sorting operation, to the second space.
